SEQUENCE LISTING (2 sequences)

SEQ ID NO: 1 - DNA, 50 bp, Artificial Sequence
  Synthetic Illumina probe for detection of PIK3CD RNA
agctctgttctgattcaccaggggtccgtcagtagtcattgccacccgcg

SEQ ID NO: 2 - DNA, 50 bp, Artificial Sequence
  Synthetic Illumina probe for detecting CX3CL1 mRNA
tttgtgaggaagccgctggggccagttggtcccccttccatggactttgt

(19) United States
(12) Patent Application Publication
(10) Pub. No.: US 2017/0335367 A1
(43) Pub. Date: Nov. 23, 2017

(54) METHOD TO PREDICT THE LACK OF RESPONSE TO ANTI-TNF ALPHA THERAPIES

(71) Applicant: Fundacio Hospital Universitari Vall D'Hebron-Institut De Recerca, Barcelona (ES)

(72) Inventors: Sara Marsal BARRIL, Barcelona (ES); Antonio Julia CANO, Barcelona (ES); Juan De Dios Canete CRESPILLO, Sabadell (ES)

(73) Assignees: Fundacio Hospital Universitari Vall D'Hebron-Institut De Recerca, Barcelona (ES); HOSPITAL CLINIC DE BARCELONA, Barcelona (ES)

(21) Appl. No.: 15/421,175

(22) Filed: Jan. 31, 2017

Related U.S. Application Data
(63) Continuation of application No. PCT/EP2014/066604, filed on Aug. 1, 2014.

(51) Int. Cl. C12Q 1/48; C07K 16/24; G01N 33/577; C12Q 1/00

(52) U.S. Cl. CPC C12Q 1/485; C12Q 1/005; C07K 16/241; G01N 33/577; C07K 2317/73; G01N 2500/00

(57) ABSTRACT

This invention provides methods for predicting response to anti-TNFα biological agent treatment in a rheumatoid arthritis patient and methods for selecting a treatment for a rheumatoid arthritis patient, the methods comprising determining the level of expression of PIK3CD as a biomarker, and optionally also determining the level of expression of CX3CL1 as a second biomarker. The invention additionally provides kits for carrying out the methods described.

The present invention provides an in vitro method to predict the lack of response to anti-TNF alpha therapies in rheumatoid arthritis patients, as well as a method to select a treatment for these patients. The method provides a new tool for personalized medicine and is applicable in the clinical management of the disease.

### BACKGROUND ART

In the recent years, recombinant proteins and antibodies have proven to be highly successful drugs for the treatment of autoimmune diseases. This has been evident in the management of Rheumatoid Arthritis (RA). RA patients are initially treated with the so-called Disease Modifying Anti-rheumatic Drugs (DMARDS). In a high proportion of patients, however, DMARDs are incapable of reducing the inflammation and slowing down the progression of the disease. In the last decade, recombinant proteins and antibodies targeted to inhibit the Tumour Necrosis Factor alpha (TNF-alpha)—a cytokine highly expressed in pathological tissue—have shown to be highly efficacious treatments in RA.

Although the treatment of RA with biologic therapies has had a tremendous positive impact on the way the disease is managed, there are a number of shortcomings associated to their use. Arguably, two of the biggest are lack of response in a subgroup of patients and treatment costs. It is estimated that around 25% of rheumatoid arthritis patients treated with any of the five biological anti-TNFalpha therapies currently available (Infliximab, Adalimumab, Golimumab, Etanercept and Certolizumab-Pegol) will not respond to this type of treatment. Moreover, compared to conventional DMARDs, anti-TNF therapies are very expensive and have a huge impact on the health systems.

Therefore there is a need in the art to find ways to predict whether a patient will respond to these biological drugs, so that those patients who are very unlikely to respond can be given alternative therapies. As result of this treatment personalization, the overall quality of life of RA patients will improve and the burden on health systems associated with their treatment will be reduced.

Most of the methods described in the art to predict response to biological DMARDs are based on the use of Rheumatoid Factor (RF) and/or anti-cyclic citrullinated peptide (anti-CCP) as markers. Their levels are determined at baseline (that is, before the biological treatment is begun) and in some cases their absence or presence (or alternatively their levels) are taken as an indication of the risk of unresponsiveness to some or all of the anti-TNFalpha therapies (see for instance Potter M., et. al. “Association of rheumatoid factor and anti-cyclic citrullinated peptide positivity, but not carriage of shared epitope or PTPN22 susceptibility variants, with anti-tumour necrosis factor response in rheumatoid arthritis”, Ann. Rheum. Dis. 2009, vol. 68, pp. 69-74 and Bobbio-Pallavicini F. et. al. “High IgA rheumatoid factor levels are associated with poor clinical response to tumour necrosis factor α inhibitors in rheumatoid arthritis” Ann. Rheum. Dis. 2007, vol. 66, pp. 302-307). Although the references cited associate high levels of these biomarkers with lack of response, others point to an opposite direction, and relate low levels to unresponsiveness (for instance Klaasen R., et. al. “The value of rheumatoid factor and anti-citrullinated protein antibodies as predictors of response to infliximab in rheumatoid arthritis: an exploratory study” Rheumatology 2011, vol. 50, pp. 1487-1493). This lack of consensus warrants the search for new and more reliable biological markers for the prediction of treatment outcome of RA.

Cytokines have also been studied as potential biomarkers for predicting disease and treatment outcomes (see Davis J M. Et. al. “Cytokine biomarkers and the promise of personalized therapy in rheumatoid arthritis” Reumatol. Clin. 2009, vol. 5, pp. 143-146). DNA seequence polymorphisms have also been associated to diagnosis, prognosis and treatment response in RA. However, the clinical applicability of these biomarkers and polymorphisms is low. Some of the biomarker studies have been performed in very small numbers of patients and, consequently, their applicability to the general population is questionable. Other biomarkers have only been developed for predicting the outcome for only one specific therapeutic agent. To date, as is discussed in McGeough C M. et. al. “Diagnostic, prognostic and theranostic genetic biomarkers for Rheumatoid Arthritis” Clin. & Cell. Immunology 2012, pp. 1-5, no biomarkers have been confirmed as robust predictors of response to anti-TNF-alpha therapies in RA.

Consequently, there is still a need in the art for new tools to predict response to anti-TNF biologic treatments in RA.

### SUMMARY OF THE INVENTION

Inventors have surprisingly found that phosphatidylinositol-4,5-bisphosphate 3-kinase, catalytic subunit delta (PIK3CD) is a very robust biomarker for predicting the response to treatment of RA with anti-TNFalpha biological therapies. Therefore, the invention proposes the use of PIK3CD as biomarker for predicting such a response. The invention adds a new tool with which to make informed decisions in the treatment of the disease.

Thus, a first aspect of the invention is an in vitro method for predicting response to anti-TNF-alpha biological inhibitor treatment in a rheumatoid arthritis patient, the method comprising: a) determining the level of expression of PIK3CD in a body tissue test sample of the patient; and b) comparing the level of expression of PIK3CD of step (a) with a reference control level of expression, wherein if the level of expression determined in step (a) is higher than the reference control level of expression, it is indicative that the patient will not respond to treatment with anti-TNFα biological inhibitors, and if the level determined in step (a) is lower than the reference control level of expression, it is indicative that the patient will respond to anti-TNFα biological inhibitor.

A second aspect of the invention is an in vitro method of selecting a treatment for a rheumatoid arthritis patient, the method comprising: a) determining the level of expression of PIK3CD in a body tissue test sample of the patient; and b) comparing the level of expression of PIK3CD of step (a) with a reference control level of expression, wherein if the level of expression determined in step (a) is higher than the reference control level of expression, the patient is not treated with an anti-TNFα biological inhibitor, and if the level determined in step (a) is lower than the reference control level of expression, the patient is treated with an anti-TNFα biological inhibitor.

Thus, with this second aspect of the invention, the RA patient is selected or recommended for starting an anti-TNF alpha biological therapy, that is, he/she is considered to be a good candidate for treatment with Infliximab, Adalimumab, Golimumab, Etanercept or Certolizumab-Pegol when the level determined in step (a) is lower than the reference control level of expression of PIK3CD. Therefore, the aspect encompasses a) determining the level of expression of PIK3CD in a body tissue test sample of the patient; and b) comparing the level of expression of PIK3CD of step (a) with a reference control level of expression, wherein if the level of expression determined in step (a) is higher than the reference control level of expression, the patient is not recommended to be treated with an anti-TNFα biological inhibitor, and if the level determined in step (a) is lower than the reference control level of expression, the patient is recommended to be treated with an anti-TNFα biological inhibitor.

A third aspect of the invention, is an in vitro method of selecting a treatment for a rheumatoid arthritis patient, the method comprising: a) determining the level of expression of PIK3CD, and additionally determining the level of expression of chemokine (C-X3-C motif) ligand 1 (CX3CL1) in a body tissue test sample of the patient; and b) calculating the ratio given by the level of expression of CX3CL1 over the level of expression of PIK3CD, wherein if said ratio is higher than a reference control ratio, then the patient is treated with an anti-TNFα biological agent, and if the ratio is lower than a reference control level then the patient is not treated with an anti-TNFα biological agent. As above indicated for the second aspect, this aspect comprises: a) determining the level of expression of PIK3CD, and additionally determining the level of expression of CX3CL1 in a body tissue test sample of the patient; and b) calculating the ratio given by the level of expression of CX3CL1 over the level of expression of PIK3CD, wherein if said ratio is higher than a reference control ratio, then the patient is recommended to be treated with an anti-TNFα biological agent, and if the ratio is lower than a reference control level then the patient is not recommended to be treated with an anti-TNFα biological agent.

With the first, second and third aspects of the invention, those RA patients that have been predicted not to respond to anti-TNF-alpha biological agents should be spared from such treatments and should be given or recommended for alternative therapies.

These three aspects of the invention can be implemented in a kit for point-of-care testing. Thus, a fourth aspect of the present invention is a kit for selecting a treatment for a rheumatoid arthritis patient, which comprises at least one agent for determining the level of expression of PIK3CD.

### BRIEF DESCRIPTION OF THE DRAWINGS

FIG. 1. Representative immunohistochemical analysis of PIK3CD-expressing cells in synovial membrane of a RA patient with good EULAR response before (A) and 5 months later (B) of anti-TNF-alpha therapy. (×20).

FIG. 2. Change in synovial PIK3CD expression after 20 weeks of anti-TNF therapy according to the EULAR response. Good and Moderate EULAR responders showed a significant decrease of synovial tissue PIK3CD/mm² protein while EULAR none responders showed an increase of this biomarker. The x axis represents the change in synovial PIK3CD/mm² (5 months), the y-axis represents EULAR GOOD (left column), EULAR MODERATE (center column) and EULAR NONE (right column).

FIG. 3. Discrimination between responder and non-responder patients by way of using the CX3CL1 expression/PIK3CD expression ratio. As can be seen by computing the ratio of expression of the two markers, a clear seggregation between responders and non responders is achieved (circles correspond to responders, squares to non-responders, and diamonds to osteoarthritis control patients).

### DETAILED DESCRIPTION OF THE INVENTION

For the sake of understanding, the following definitions are included.

The term “anti-TNFα (or anti-TNF-alpha) biological agent treatment” as used herein indicates a treatment based on the administration of any of the biological anti-TNFα drugs currently available in the market, that is: the monoclonal antibodies Infliximab (trade name Remicade), Adalimumab (trade name Humira) and Golimumab (trade name Simponi), the monoclonal antibody fragment Certolizumab-Pegol (trade name Cimzia) and the fusion recombinant protein Etanercept (trade name Enbrel).

The term “predicting response to anti-TNFα biological agent treatment” as used herein refers to the determination of the likelihood that the patient will respond to the administered anti-TNFα biological agent treatment. The response to these treatments can be measured by applying the European League Against Rheumatism (EULAR) response criteria. The EULAR (European League Against Rheumatism) response criteria classifies the patient individuals as non-, moderate or good responders depending on the change and the level of the Disease Activity Score (DAS).

The term “reference control level of expression” referred to in the methods of the invention is to be understood as a predefined value of a given molecular marker, PIK3CD (and optionally CX3CL1) in the present case, which is (are) derived from the levels of said molecular marker in a sample or group of samples. The samples are taken from a patient or group of patients wherein the presence, absence, stage, or course of the disease has properly been determined previously. The patient or patients from whom the “reference control level of expression” is derived may include patients wherein the condition is absent, patient/s wherein the condition is present, or both. The person skilled in the art, making use of the general knowledge, is able to choose the patient or group of patients more adequate for obtaining the control levels for each of the methods of the present invention. Methods for obtaining the reference value from the group of patients selected are well-known in the state of the art (Burtis C. A. et al., 2008, Chapter 14, section “Statistical Treatment of Reference Values”). In a particular case the “reference control level of expression” for PIK3CD (and optionally CX3CL1) is a cut-off value defined by means of a conventional ROC analysis (Receiver Operating Characteristic analysis). As the skilled person will appreciate, optimal cut-off values will be defined according to the particular application of the method, target population for the prediction, balance between specificity and sensitivity, experimental techniques used in its definition, and others.

The term “reference control ratio” is to be understood here as a predefined value of the level of expression of CX3CL1 over the level of expression of PIK3CD. This ratio is derived from the levels of both markers in a sample or group of samples. The samples are taken from a patient or group of patients wherein the presence, absence, stage, or course of the disease has properly been determined previously. The patients may include patients wherein the condition (or the response to treatment) is absent, present, or both. It might be obtained at the mRNA or protein levels. As the skilled person will appreciate, the ratio may vary depending on the experimental techniques used to calculate de level of expression of both markers.

The term “body tissue test sample” is to be understood as tissue or a liquid originating from inside the living body. It includes tissues and fluids that are excreted or secreted from the body. In particular, in the present application, by tissue it is to be understood both a solid tissue such as synovial tissue and also a liquid tissue such as blood or plasma.

The term “antibody or a fragment thereof binds to the protein coded by the PIK3CD gene” and the term “antibody or a fragment thereof which is capable of binding to the protein coded by the PIK3CD gene” are to be understood here as any immunoglobulin or fragment thereof able to selectively bind the PIK3CD gene product. It includes monoclonal and polyclonal antibodies. The term “fragment thereof” encompasses any part of an antibody having the size and conformation suitable to bind an epitope of PIK3CD. Suitable fragments include F(ab), F(ab′), Fv and nanobodies, among others. An “epitope” is the part of the antigen being recognized by the immune system (B-cells, T-cells or antibodies).

The term “antibody or a fragment thereof binds to the protein coded by the CX3CL1 gene” and the term “antibody or a fragment thereof which is capable of binding to the protein coded by the CX3CL1 gene” are to be understood here as any immunoglobulin or fragment thereof able to selectively bind the CX3CL1 gene product. It includes monoclonal and polyclonal antibodies. The term “fragment thereof” encompasses any part of an antibody having the size and conformation suitable to bind an epitope of CX3CL1. Suitable fragments include F(ab), F(ab′), Fv and nanobodies, among others. An “epitope” is the part of the antigen being recognized by the immune system (B-cells, T-cells or antibodies).

PIK3CD is used herein as an abbreviation of human phosphatidylinositol-4,5-bisphosphate 3-Kinase catalytic subunit delta (also known as pI3K-delta or p110-delta). The protein corresponds to the O00329 entry of the Uniprot database (last modified Dec. 11, 2013). PIK3CD belongs to the family of phosphatidylinositol kinases, and is expressed mainly in leukocytes.

CX3CL1 is used herein as an abbreviation of human CX3CR1 Ligand 1 (also known as fractalkine or C-X3-C motif chemokine 1). The protein corresponds to the entry P78423 of the Uniprot database (last modified Nov. 13, 2013). It is a ligand for the human CX3CR1 chemokine receptor expressed in hematopoietic cells. It has been shown to participate in the chemotaxis of T cells and monocytes.

As mentioned above, a first aspect of the invention is an in vitro method for predicting response to anti-TNF-alpha biological agent treatment in a rheumatoid arthritis patient, the method comprising: a) determining the level of expression of PIK3CD in a body tissue test sample of the patient; and b) comparing the level of expression of PIK3CD of step (a) with a reference control level of expression, wherein if the level of expression determined in step (a) is higher than the reference control level of expression, it is indicative that the patient will not respond to treatment with anti-TNF-alpha biological agents, and if the level determined in step (a) is lower than the reference control level of expression, it is indicative that the patient will respond to anti-TNF-alpha biological agent.

In a particular embodiment of the first aspect of the invention, the reference control level of expression for PIK3CD is 7.5 when the techniques used for the detection are those disclosed in the present description (Examples section).

In a particular embodiment of the first aspect of the invention, optionally in combination with any of the embodiments below, the in vitro method comprises: a) determining the level of expression of PIK3CD, and additionally determining the level of expression of CX3CL1 in a body tissue test sample of the patient; and b) comparing the level of expression of PIK3CD of step (a) with its reference control level of expression, wherein if the level of expression determined in step (a) is higher than its reference control level of expression, it is indicative that the patient will not respond to treatment with anti-TNF-alpha biological agents, and if the level determined in step (a) is lower than its reference control level of expression, it is indicative that the patient will respond to anti-TNF-alpha biological agents, and additionally comparing the level of expression of CX3CL1 of step (a) with its reference control level of expression, wherein if the level of expression determined in step (a) for CX3CL1 is higher than its reference control level of expression, it is indicative that the patient will respond to treatment with anti-TNF-alpha biological agents, and if the level determined in step (a) for CX3CL1 is lower than its reference control level of expression, it is indicative that the patient will not respond to anti-TNF-alpha biological agents.

In a particular embodiment of the first aspect of the invention, the reference control level of expression for PIK3CD is 7.5 and the reference control level of expression for CX3CL1 is 6.7 when the techniques used for the detection are those disclosed in the present description (Examples section).

As also mentioned above, a second aspect of the invention is an in vitro method of selecting a treatment for a rheumatoid arthritis patient, the method comprising: a) determining the level of expression of PIK3CD in a body tissue test sample of the patient; and b) comparing the level of expression of PIK3CD of step (a) with a reference control level of expression, wherein if the level of expression determined in step (a) is higher than the reference control level of expression, the patient is not treated (or not recommended to be treated) with an anti-TNF-alpha biological agent, and if the level determined in step (a) is lower than the reference control level of expression, the patient is treated (or recommended to be treated) with an anti-TNF-alpha biological agent.

In a particular embodiment of the second aspect of the invention, the reference control level of expression for PIK3CD is 7.5 when the techniques used for the detection are those disclosed in the present description (Examples section).

In a particular embodiment of the second aspect of the invention, optionally in combination with any of the embodiments above or below, the in vitro method, comprises: a) determining the level of expression of PIK3CD, and additionally determining the level of expression of CX3CL1 in a body tissue test sample of the patient; and b) comparing the level of expression of PIK3CD of step (a) with its reference control level of expression, and additionally comparing the level of expression of CX3CL1 of step (a) with its reference control level of expression, wherein if the level of expression determined in step (a) for PIK3CD is higher than its reference control level of expression and the level of expression determined in step (a) for CX3CL1 is lower than its reference control level of expression then the patient is not treated (or not recommended to be treated) with an anti-TNF-alpha biological agent, and if the level of expression determined in step (a) for PIK3CD is lower than its reference control level of expression and the level of expression determined in step (a) for CX3CL1 is higher than its reference control level of expression, then the patient is treated (or recommended to be treated) with an anti-TNF-alpha biological agent.

In a particular embodiment of the second aspect of the invention, the reference control level of expression for PIK3CD is 7.5 and the reference control level of expression for CX3CL1 is 6.7 when the techniques used for the detection are those disclosed in the present description (Examples section).

As also mentioned above, a third aspect of the invention, is an in vitro method of selecting a treatment for a rheumatoid arthritis patient, the method comprising: a) determining the level of expression of PIK3CD, and additionally determining the level of expression of CX3CL1 in a body tissue test sample of the patient; and b) calculating the ratio given by the level of expression of CX3CL1 over the level of expression of PIK3CD, wherein if said ratio is higher than a reference control ratio, then the patient is treated (or recommended to be treated) with an anti-TNFα biological agent, and if the ratio is lower than a reference control level then the patient is not treated (or not recommended to be treated) with an anti-TNFα biological agent.

In a particular embodiment of the third aspect of the invention, the reference control ratio is 0.914, and therefore, the patients with a ratio above this reference control ratio will respond to treatment with anti-TNFα biological agents, whereas the patients with a ratio below this reference control ratio will not respond to treatment with anti-TNFα biological agents.

In a particular embodiment of the first, second and third aspects of the invention, optionally in combination with any of the embodiments above or below, the sample is selected from the group consisting of synovial tissue or fluid, plasma, blood serum or whole blood.

In a particular embodiment of the first, second and third aspects of the invention, optionally in combination with any of the embodiments above or below, the level of expression is determined at the mRNA level.

In another particular embodiment of the first, second and third aspects, optionally in combination with any of the embodiments above or below, the determination of the mRNA level of PIK3CD or the determination of the mRNA level of CX3CL1 comprise a step wherein a nucleic acid probe is hybridized to the mRNA of PIK3CD or to the mRNA of CX3CL1 respectively.

In another particular embodiment of the first, second and third aspects, optionally in combination with any of the embodiments above or below, the determination of the mRNA level of PIK3CD comprises the use of a probe with the SEQ ID NO.1, and the determination of the mRNA level of CX3CL1 comprises the use of a probe with the SEQ ID NO.2. For a “probe with a particular sequence” it is to be understood a probe comprising or consisting of said particular sequence.

In a particular embodiment of the first, second and third aspects of the invention, optionally in combination with any of the embodiments above or below, the level of expression is determined at the protein level, which means that expressed proteins are detected

In another particular embodiment of the first, second and third aspects of the invention, optionally in combination with any of the embodiments above or below, the determination of the protein level of PIK3CD or the determination of the protein level of CX3CL1 comprise a step wherein an antibody or a fragment thereof binds to the protein coded by the PIK3CD gene or to the protein coded by the CX3CL1 gene respectively.

The nucleic acid probe to be hybridized to the mRNA of PIK3CD or alternatively the nucleic acid probe to be hybridized to the mRNA of CX3CL1, and also the antibody or fragment thereof for detecting PIK3CD and alternatively the antibody or fragment thereof for detecting CX3CL1 can be included in a kit. The kit may additionally comprise means (additives, solvents) to visualize the antigen-antibody interactions (dipsticks, chemiluminescent reagents, turbidimetric reagents, etc.).

As also mentioned above, a fourth aspect of the present invention is a kit for selecting a treatment for a rheumatoid arthritis patient, which comprises at least one agent for determining the level of expression of PIK3CD.

The kit could also be used for the method of the first aspect of the invention, that is, for predicting response to anti-TNF-alpha biological agent treatment in a rheumatoid arthritis patient.

In a particular embodiment of the fourth aspect of the invention, optionally in combination with any of the embodiments above or below, the kit comprises at least one agent for determining the level of expression of PIK3CD which is capable of hybridizing to a partial sequence of the PIK3CD mRNA. In a most particular embodiment, the agent is a nucleic acid probe that hybridizes to the mRNA of PIK3CD. In yet a most particular embodiment, the probe comprises SEQ ID NO: 1. In another particular embodiment, also optionally in combination with any of the embodiments above or below, the probe consists in SEQ ID NO: 1.

In another particular embodiment of the fourth aspect of the invention, optionally in combination with any of the embodiments above or below, the kit comprises at least one agent for determining the level of expression of PIK3CD, the agent being an antibody or a fragment thereof which is capable of binding to the protein coded by the PIK3CD gene.

In another particular embodiment of the fourth aspect of the invention, optionally in combination with any of the embodiments above or below, the kit further comprises at least one agent for determining the level of expression of CX3CL1.

In another particular embodiment of the fourth aspect of the invention, optionally in combination with any of the embodiments above or below, the kit comprises at least one agent for determining the level of expression of PIK3CD and at least one agent for determining the level of expression of CX3CL1, being both agents capable of hybridizing to a partial sequence of the PIK3CD mRNA and to a partial sequence of the CX3CL1 mRNA respectively. In a most particular embodiment, the agent capable of hybridizing to a partial sequence of the CX3CL1 mRNA is a nucleic acid probe that hybridizes to the mRNA CX3CL1. In yet a most particular embodiment, the probe comprises SEQ ID NO: 2. In another particular embodiment, also optionally in combination with any of the embodiments above or below, the probe consists in SEQ ID NO: 2.

In another particular embodiment of the fourth aspect of the invention, optionally in combination with any of the embodiments above or below, the kit comprises at least one agent for determining the level of expression of PIK3CD and at least one agent for determining the level of expression of CX3CL1, being both agents antibodies or fragments thereof which are capable of binding to the protein coded by the PIK3CD gene and to the protein coded by the CX3CL1 gene respectively.

In another particular embodiment of the fourth aspect of the invention, optionally in combination with any of the embodiments above or below, the kit comprises electronic means for calculating the level of expression of CX3CL1 and the level of expression of PIK3CD, and optionally for calculating the ratio given by the level of expression of CX3CL1 over the level of expression of PIK3CD.

These electronic means may comprise a computer readable memory comprising data linking the level of expression of PIK3CD and optionally of CX3CL1 with prediction response to anti-TNF-alpha biological agent treatment. Suitable equipment may read the memory and be able to indicate directly the response prediction and/or the selection of a treatment.

Any of the herewith disclosed in vitro methods, having in common that all give data about the response to anti-TNF-alpha biological agent treatments using PIK3CD and optionally CX3CL1 as biomarkers, may in any particular embodiment or combination of embodiments include a further step of collecting and/or providing and/or saving data derived from previous steps in a data carrier. Thus, the invention also encompasses any data carrier with the predicted response or selected treatment data directly obtained from any of the methods of the invention.

In the sense of the invention a “data carrier” is to be understood as any means that contain meaningful information data for the prediction of response to anti-TNF-alpha with the determined level of expression of PIK3CD and optionally with the determined level of expression of CX3CL1. Examples of data carrier are printed copies of paper with the determined levels of PIK3CD and optionally of CX3CL1 determined in the synovial fluid or tissue, plasma, blood serum or whole blood according to these methods, and correlating with the prediction of response. The carrier may also be any entity or device capable of carrying the prognosis data. For example, the carrier may comprise a storage medium, such as a ROM, for example a CD ROM or a semiconductor ROM, or a magnetic recording medium, for example a floppy disc or hard disk. Further, the carrier may be a transmissible carrier such as an electrical or optical signal, which may be conveyed via electrical or optical cable or by radio or other means. When the prognosis data are embodied in a signal that may be conveyed directly by a cable or other device or means, the carrier may be constituted by such cable or other device or means. Other carriers relate to USB devices and computer archives.

Throughout the description and claims the word “comprise” and variations of the word, are not intended to exclude other technical features, additives, components, or steps. Furthermore, the word “comprise” and its variations encompasses the term “consisting of”. Additional objects, advantages and features of the invention will become apparent to those skilled in the art upon examination of the description or may be learned by practice of the invention. The following examples are provided by way of illustration, and they are not intended to be limiting of the present invention. Furthermore, the present invention covers all possible combinations of particular and preferred embodiments described herein.

### EXAMPLES

### A) Patients and Methods

### Patients and Samples

Eleven patients (8 women, 3 men) fulfilling the American College of Rheumatology/European League Against Rheumatism 2010 criteria for RA (Aletaha, D. et. al. “2010 Rheumatoid arthritis classification criteria: An American College of Rheumatology/European League Against Rheumatism collaborative initiative” Arth. Rheumatol. 2010, vol. 62, pp. 2569-2581), with a basal mean Disease Activity Score in 28 joints (DAS28) (Prevoo M L., et. al. “Modified disease activity scores that include twenty-eight joint counts. Development and validation in a prospective longitudinal study of patients with rheumatoid arthritis” Arthritis Rheum. 1995, vol. 38, pp. 44-48) of 5.3 [4.2-6.9]) (median and interquartile range [IQR]), for whom an anti-TNF was prescribed by their rheumatologist (n=6 infliximab, n=3 adalimumab, n=2 etanercept), were enrolled in this study. Response to treatment defined by the EULAR response criteria (Fransen J., et. al. “The Disease Activity Score and the EULAR response criteria” Rheum. Dis. Clin. North. Am. 2009, vol. 35, pp. 745-757, vii-viii) was determined after 20 weeks of therapy, aggregating moderate and good responders into a single responder group. The study was approved by the local ethics committee and all the patients signed the informed consent. This study was conducted in accordance with the Declaration of Helsinki principles.

Synovial samples were obtained by guided arthroscopy using a 2.7 mm arthroscope (Storz, Tuttlingen, Germany) under local anesthesia. In all patients, 6-8 biopsies were taken from the suprapatellar pouch and medial and lateral gutters with a 3 mm grasping forceps before starting a TNF antagonist. All patients underwent a second arthroscopy with synovial sampling after 20 weeks of therapy. Synovial biopsies for mRNA analysis were immediately stored in RNALater preserving agent (QIAGEN, USA) and frozen to −80° C. until RNA extraction (Van de Sande M G., et. al. “Evaluating anthrheumatic treatments using synovial biopsy: a recommendation for standardisation to be used in clinical trials” Ann. Rheum. Dis. 2011, vol. 70, pp. 432-437).

### Microarray Analysis

RNA was extracted from synovial biopsies using RNA Mini Kit (Qiagen, US) and the integrity was assessed using BioAnalyzer microfluidic gel analysis (Agilent, USA). All samples were of high quality (RNA Integrity Number >8) and were subsequently analyzed using Sentrix whole genome Beadchips WG6 version 2 (Illumina, US). Briefly, after RNA isolation, biotin-labeled cRNA was prepared using the Ambion Illumina RNA amplification kit (Ambion, US) amd Illumina TotalPrep RNA Amplification Kit (Ambion, US). Biotin-labeled cRNA (1.5 μg) was hybridized to WG6 Beadchips and scanned on the 500× Illumina BeadStation. Data collection was performed using BeadStudio 3.1.1.0 software (Illumina, US). Raw and normalized data will be available at NCBI GEO database with accession number GSE47726.

The probe used for the detection of the
expression of PIK3CD in the Illumina
microarray was SEQ ID NO 1:
AGCTCTGTTCTGATTCACCAGGGGTCCGTCAGTAGTCATTGCCAC
CCGCG
The probe used for the detection of the
expression of CX3CL1 in the Illumina
microarray was SEQ ID NO 2:
TTTGTGAGGAAGCCGCTGGGGCCAGTTGGTCCCCCTTCCATGGAC
TTTGT

In order to use the most recent human genome annotation information, only microarray probes matching curated gene sequences from NCBI RefSeq database (release 51) (Pruitt K D. et. al. “NCBI reference sequence project: update and current status” Nucl. Acids Res. 2003, vol. 31, pp. 34-37) were selected. A number of 21,189 curated probes representing the expression of 18,524 different human genes were finally selected for analysis.

After log 2 transformation, the raw gene expression data was quantile-normalized. The presence of a potential bias between batches of microarrays was minimized using the Bayesian ComBat procedure (Johnson W E. et. al. “Adjusting batch effects in microarray expression data using empirical Bayes methods” Biostatistics 2007, vol. 8, pp. 118-127). The differential gene expression between responders and non-responders was calculated using the t-test implemented in the multitest package from the R statistical software (http://www.R-project.org/). In order to control for the presence of false positives, the Bonferroni multiple test correction was applied to the nominal P-values. In order to characterize the presence of significant functional features in the group of differentially expressed genes, we analyzed the results using DAVID Functional Annotation Tool (Huang da W. et. al. “Systematic and integrative analysis of large gene lists using DAVID bioinformatics resources” Nat. Protoc. 2009, vol. 8, pp. 44-57). Briefly, DAVID annotation tool uses curated biological knowledge from multiple databases to explore the overrepresentation of functional features. Using a list of genes of interest such as those overexpressed in anti-TNF non-responders, DAVID identifies the associated biological features and it tests its abundance over the frequency of the same features in the global human dataset.

Real-Time PCR analysis of the genes most significantly associated with treatment response was performed using the Taqman platform (Life Technologies, US). Taqman FAM-labeled assays were used to measure the expression of PIK3CD (Hs00192399_m1) and CX3CL1 (Hs00171086_m1) genes. The Ribosomal Protein L11 VIC-labeled assay (Hs00831112_s1) was used as an endogenous control gene, and all assays were performed using the Taqman Universal PCR master mix (Life Technologies, US). Gene expression analysis was performed using the recommended protocol with the Applied Biosystems HT7900 system (ABI, US) and fold-change expression was calculated from the Ct values of the test gene and the endogenous gene using the −2ΔΔCt method. Correlation between microarray gene expression values and RT-PCR values was calculated using Pearson's product moment correlation.

### Immunohistochemistry and Digital Image Analysis

Four synovial samples of each patient were fixed in 1% formaldehyde and embedded in paraffin, sectioned and subjected to antigen retrieval as detailed elsewhere (Canete J D., et. al. “Clinical significance of synovial lymphoid neogenesis and its reversal after anti-tumour necrosis factor alpha therapy in rheumatoid arthritis” Ann. Rheum. Dis. 2009, vol. 68, pp. 751-756). The slides were subsequently stained with an automated immunostainer (TechMate 500 Plus; Dako, Cambridge, UK) using the following monoclonal antibodies: PI 3-kinase p110δ (1/25) (A-8) (Santa Cruz Biotechnology, Inc., San Diego, Calif., USA) and anti-CD68 (clone KP-1; Dako). As a negative control, the primary antibodies were substituted by isotype- and concentration matched control antibodies. The primary antibodies were subsequently detected by a avidin-biotin-peroxidase-based method (Envision System, Dako) and a aminoethylcarbazole color reaction (Sigma-Aldrich, St. Louis, Mont., USA) as described previously in detail. Finally, the slides were counterstained with hematoxylin.

Slides were scored by Digital Image Analysis (DIA) by an independent observer who was blinded to the clinical data. All sections in each slide were systematically counted, with a mean of 57 sections (range 23 to 88), using the AnalySIS® Image Processing Program (Olympus®, Tokyo, Japan) (12). Positive staining of cellular markers was expressed as positive cells/mm2.

### B) Results

A total of 8 patients responded to TNF blockade, with an average DAS28 reduction of 1.6 (P=0.039), while 3 patients did not show a significant clinical response (P=0.56) according to EULAR response criteria. Clinical and demographic data of RA patients are detailed in Table 1.

TABLE 1
Clinical and demographical characteristics of the RA patient cohort

| Demographics | |
|---|---|
| Women n (%) | 8 (72.7%) |
| Age (years) | 60.3 (51.1; 71.6) |
| Disease duration (years) | 17.0 (0.7; 39.2) |
| Follow-up (years) | 2.6 (0.3; 4.5) |
| DAS28 3v (0 week) | 5.90 (3.91-8.11) |
| DAS28 3v (20 week) | 4.58 (2.98-7.39) |
| ACPA (UI) (n = 10) | 82% |
| EULAR response | |
| Good and Moderate n (%) | 8 (72.7%) |
| No Response n (%) | 3 (27.3%) |

### Microarray Analysis of Synovial Tissue

Differential gene expression analysis between ST samples of responders and non-responders identified a total of n=153 genes significantly associated with treatment response after Bonferroni multiple test correction. From these, n=76 were overexpressed in the non-responder group and n=57 were overexpressed in the anti-TNF responder group (Table 2).

TABLE 2
List of genes differentially expressed between anti-TNF responders and non-responders

| | | | | |
|---|---|---|---|---|
| Gene Symbol | Accession | Definition | Fold Change | P-value |

Genes significantly overexpressed in non-responders

| | | | | |
|---|---|---|---|---|
| PIK3CD | NM_005026.2 | Phosphoinositide-3-kinase, catalytic, | −1.56 | 7.11E−18 |
| | | delta polypeptide | | |
| REEP4 | NM_025232.2 | Receptor accessory protein 4 | −1.50 | 7.14E−17 |
| HCLS1 | NM_005335.3 | Hematopoietic cell-specific Lyn substrate 1 | −1.63 | 4.38E−09 |
| GCDH | NM_013976.2 | Glutaryl-Coenzyme A dehydrogenase | −1.50 | 3.2E−07 |
| ADA | NM_000022.2 | Adenosine deaminase | −1.73 | 7.15E−07 |
| NT5DC2 | NM_022908.1 | 5′-nucleotidase domain containing 2 | −1.86 | 7.7E−07 |
| SERPINH1 | NM_001235.2 | Serpin peptidase inhibitor, clade H, member 1 | −1.66 | 1.67E−06 |

Genes Significantly overexpressed in responders

| | | | | |
|---|---|---|---|---|
| CX3CL1 | NM_002996.3 | chemokine (C—X3—C motif) ligand 1 | 1.94 | 7.4E−12 |
| PLS3 | NM_005032.3 | plastin 3 (T isoform) | 1.71 | 1.97E−10 |
| DIXDC1 | NM_033425.1 | DIX domain containing 1 | 1.90 | 9.29E−10 |
| TMOD1 | NM_003275.1 | tropomodulin 1 | 2.10 | 1.56E−08 |
| PCOLCE2 | NM_013363.2 | Procollagen C-endopeptidase enhancer 2 | 2.45 | 3.34E−07 |
| PPP1R3C | NM_005398.3 | protein phosphatase 1, regulatory (inhibitor) | 2.13 | 3.64E−07 |
| | | subunit 3C | | |
| CRTAP | NM_006371.3 | cartilage associated protein | 1.58 | 4.78E−07 |
| PRELP | NM_002725.3 | proline/arginine-rich end leucine-rich repeat | 2.02 | 6.42E−07 |
| | | protein, transcript variant 1 | | |
| DKFZP686A01247 | NM_014988.1 | hypothetical protein | 2.30 | 7.13E−07 |
| UNQ689 | NM_212557.1 | RSTI689 | 5.09 | 9.54E−07 |
| OR2A9P | NR_002157.1 | olfactory receptor, family 2, subfamily A, | 2.01 | 1.94E−06 |
| | | member 9 | | |

The list of genes that are significant after Bonferroni correction and that show a >1.5 fold change between the two groups are shown.

The functional analysis of the genes overexpressed in non-responders over the gene expression profiles of 82 different human tissues (Su A I., et. al. “A gene atlas of the mouse and human protein-encoding transcriptomes” Proc. Natl. Acad. Sci. USA 2004, vol. 101, pp. 6062-6067) revealed a highly significant similarity with peripheral blood CD14+ monocyte gene expression (kappa similarity statistic=1, P=3.56E-05, P_{corrected}=2.7E-3). A total of 50 genes (69.4%) were found to be common between the synovial tissue of anti-TNF non-responders and the transcriptional profile of normal human monocytes. The same analysis over the set of genes associated with anti-TNF response did not identify a functional feature significantly overrepresented in this group.

Although it is difficult to establish a cutoff value to separate responders from non-responders, as it depends on many variables susceptible of change between experimental techniques, the values that would emanate from the study that inventors carried out would be: 7.5 for PIK3CD and 6.7 for CX3CL1 (as stated above, these are obtained after log 2 transformation and quantile-normalization).

### RT-PCR Validation

In order to perform RT-PCR validation, we used a >1.5 absolute fold change filter over the set of significantly associated genes. Two genes, one per anti-TNF response group, were finally selected. PIK3CD gene was found to be highly overexpressed in non-responder patients (P=7.11E-18, fold-change=1.56), while CX3CL1 was found to be strongly associated with treatment response after 20 weeks of therapy (P=7.4E-12, fold-change=−1.94). The RT-PCR analysis of these two genes associated with anti-TNF response showed a strong correlation with the microarray data (P=2.42E-10 and P=2.11E-11 for the expression of PIK3CD and CX3CL1 genes, respectively). Consequently, we also replicated the significant association of both genes with anti-TNF response observed with the microarray data (P<0.01, same direction effect).

### Immunohistochemical Validation of PIK3CD Synovial Expression

While CX3CL1 implication in RA has been intensively studied for more than 10 years, the evidence implicating PIK3CD in the pathogenesis of RA is very recent (Bartok B. et. al. “PI3 kinase delta is a key regulator of synoviocyte function in rheumatoid arthritis” Am. J. Pathol. 2012, vol. 180, pp. 1906-1916).

For this reason, and in order to increase our understanding in the role of this protein, we investigated by immunohistochemistry and Digital Image Analysis (DIA) its expression in ST samples obtained before and after 20 weeks of anti-TNF treatment.

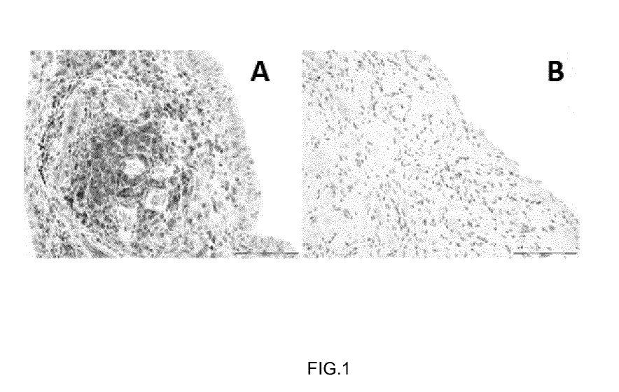

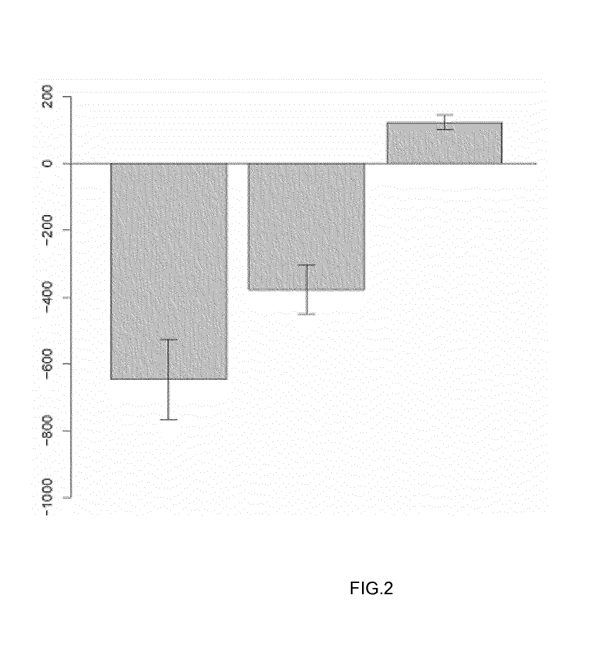

PIK3CD was found to be strongly expressed in lining and sublining cells, endothelial cells and lymphocyte aggregates (FIG. 1), confirming recent observations (Bartok B., et. al., ibid). After 20 weeks of anti-TNF-alpha therapy PIK3CD expression showed a significant reduction only in patients with EULAR response, reflecting a good negative correlation between the grade of EULAR response and the reduction in PIK3CD expression (FIG. 2 and Table 3 shown below). Non-responsive patients had a higher DAS28 at baseline (median 6.9 (IQ 5-7.7)), but similar PIK3CD/mm² and CD68/mm² count.

TABLE 3
Changes in disease activity and in PIK3CD and CD68 Synovial expression at baseline and after
20 weeks of anti-TNF therapy. Data represent number of positive cells for each protein
per mm², and are expressed in median and Inter Quartile Range (IQR).

| | All | | | Good and Moderate | | |
|---|---|---|---|---|---|---|
| | n = 11 | | | n = 8 | | |
| | 0 week | 20 week | P-value | 0 week | 20 week | P-value |
| DAS28 3v | 5.8 | 4.5 | 0.048 | 5.6 | 4.0 | 0.039 |
| VSG | (5.1-7.3) | (3.6-6.1) | | (4.8-7.1) | (3.4-5.2) | |
| PIK3CD/ | 394.2 | 62.9 | 0.071 | 576.4 | 48.5 | 0.036 |
| mm² | (89.5-829.8) | (5.8-293.7) | | (281.5-859.3) | (4.7-269.5) | |
| CD68/mm² | 1365.0 | 247.0 | 0.004 | 1503.0 | 232.0 | 0.027 |
| SL | (841.0-2769.0) | (112.0-415.0) | | (881.0-2700.0) | (119.8-658.3) | |

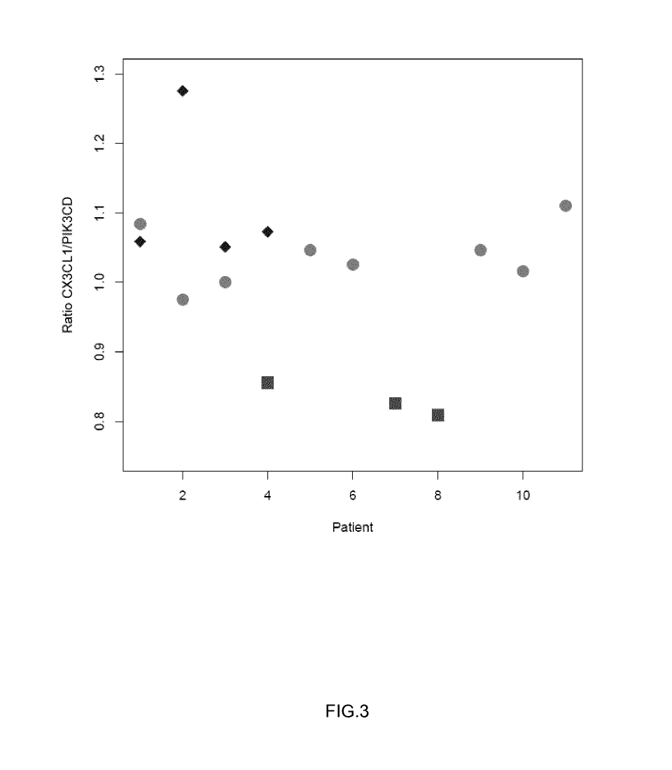

As it has been stated above, it is also a part of the invention a method based on the ratio of expression between the two markers (CX3CL1/PIK3CD). This method has the advantage of being less prone to be influenced by the experimental techniques used for measuring the level of expression in any way (be it protein or mRNA). As it can be seen in FIG. 3 there is a clear discrimination between responder and non-responder patients when the ratio of CX3CL1 expression over the expression of PIK3CD is plotted. Inspection of FIG. 3 clearly reveals that the cutoff must be near 0.9, when the experimental techniques used for the determination of expression are those disclosed in the present description.

It must be borne in mind that different cutoffs will be obtained for separating responders and non-responders depending not only on the experimental techniques used for detecting and quantifying the expression of both markers, but also on the mathematical criteria implemented for the definition of the cutoff.

One of the criteria to set the cutoff value for any of the methods of the invention could be to select the intermediate point between 2 standard deviations from the mean, of the expression of each one of the two groups (responders and non-responders). This implies that, whatever the method of the invention is (based on PIK3CD expression exclusively, based on PIK3CD expression plus CX3CL1 expression, or the ratio CX3CL1/PIK3CD), one might first obtain the mean for responders and non-responders. Then, one might obtain the value which is 2 standard deviations away from the mean for each one of the groups (responders and non-responders). The cutoff would be the value falling between those two latter values. This means that, the probability of there being any expression in this intermediate point is 5% (irrespective of the patient being responder or non-responder). With this criterium, the cutoff values for the different methods of the invention would then be: 7.5 for PIK3CD, 6.7 for CX3CL1, and 0.914 for the ratio of expression of CX3CL1/PIK3CD.

### REFERENCES CITED IN THE APPLICATION

Potter M., et. al. “Association of rheumatoid factor and anti-cyclic citrullinated peptide positivity, but not carriage of shared epitope or PTPN22 susceptibility variants, with anti-tumour necrosis factor response in rheumatoid arthritis”, Ann. Rheum. Dis. 2009, vol. 68, pp. 69-74.
Bobbio-Pallavicini F. et. al. “High IgA rheumatoid factor levels are associated with poor clinical response to tumour necrosis factor alpha inhibitors in rheumatoid arthritis” Ann. Rheum. Dis. 2007, vol. 66, pp. 302-307.
Klaasen R., et. al. “The value of rheumatoid factor and anti-citrullinated protein antibodies as predictors of response to infliximab in rheumatoid arthritis: an exploratory study” Rheumatology 2011, vol. 50, pp. 1487-1493.
Davis J M. Et. al. “Cytokine biomarkers and the promise of personalized therapy in rheumatoid arthritis” Reumatol. Clin. 2009, vol. 5, pp. 143-146.
McGeough C M. et. al. “Diagnostic, prognostic and theranostic genetic biomarkers for Rheumatoid Arthritis” Clin. & Cell. Immunology 2012, pp. 1-5.
Aletaha, D. et. al. “2010 Rheumatoid arthritis classification criteria: An American College of Rheumatology/European League Against Rheumatism collaborative initiative” Arth. Rheumatol. 2010, vol. 62, pp. 2569-2581
Prevoo M L., et. al. “Modified disease activity scores that include twenty-eight joint counts. Development and validation in a prospective longitudinal study of patients with rheumatoid arthritis” Arthritis Rheum. 1995, vol. 38, pp. 44-48.
Fransen J., et. al. “The Disease Activity Score and the EULAR response criteria” Rheum. Dis. Clin. North. Am. 2009, vol. 35, pp. 745-757, vii-viii.
Van de Sande M G., et. al. “Evaluating anthrheumatic treatments using synovial biopsy: a recommendation for standardisation to be used in clinical trials” Ann. Rheum. Dis. 2011, vol. 70, pp. 432-437.
Pruitt K D. et. al. “NCBI reference sequence project: update and current status” Nucl. Acids Res. 2003, vol. 31, pp. 34-37.
Johnson W E. et. al. “Adjusting batch effects in microarray expression data using empirical Bayes methods” Biostatistics 2007, vol. 8, pp. 118-127.
Huang da W. et. al. “Systematic and integrative analysis of large gene lists using DAVID bioinformatics resources” Nat. Protoc. 2009, vol. 8, pp. 44-57.
Canete J D., et. al. “Clinical significance of synovial lymphoid neogenesis and its reversal after anti-tumour necrosis factor alpha therapy in rheumatoid arthritis” Ann. Rheum. Dis. 2009, vol. 68, pp. 751-756.
Su A I., et. al. “A gene atlas of the mouse and human protein-encoding transcriptomes” Proc. Natl. Acad. Sci. USA 2004, vol. 101, pp. 6062-6067.
Bartok B. et. al. “PI3 kinase delta is a key regulator of synoviocyte function in rheumatoid arthritis” Am. J. Pathol. 2012, vol. 180, pp. 1906-1916.

1. An in vitro method for predicting response to anti-TNFα biological agent treatment in a rheumatoid arthritis patient, the method comprising:
a) determining the level of expression of PIK3CD in a body tissue test sample of the patient; and
b) comparing the level of expression of PIK3CD of step (a) with a reference control level of expression, wherein if the level of expression determined in step (a) is higher than the reference control level of expression, it is indicative that the patient will not respond to treatment with anti-TNFα biological agents, and if the level determined in step (a) is lower than the reference control level of expression, it is indicative that the patient will respond to anti-TNFα biological agent.

2. The in vitro method for predicting response to anti-TNFα biological agent treatment according to claim 1, the method comprising:
a) determining the level of expression of PIK3CD, and additionally determining the level of expression of CX3CL1 in a body tissue test sample of the patient; and
b) comparing the level of expression of PIK3CD of step (a) with its reference control level of expression, wherein if the level of expression determined in step (a) is higher than its reference control level of expression, it is indicative that the patient will not respond to treatment with anti-TNFα biological agent, and if the level determined in step (a) is lower than its reference control level of expression, it is indicative that the patient will respond to anti-TNFα biological agent, and additionally comparing the level of expression of CX3CL1 of step (a) with its reference control level of expression, wherein if the level of expression determined in step (a) for CX3CL1 is higher than its reference control level of expression, it is indicative that the patient will respond to treatment with anti-TNFα biological agent, and if the level determined in step (a) for CX3CL1 is lower than its reference control level of expression, it is indicative that the patient will not respond to anti-TNFα biological agent.

3. An in vitro method of selecting a treatment for a rheumatoid arthritis patient, the method comprising:
a) determining the level of expression of PIK3CD in a body tissue test sample of the patient; and
b) comparing the level of expression of PIK3CD of step (a) with a reference control level of expression, wherein if the level of expression determined in step (a) is higher than the reference control level of expression, the patient is not treated with an anti-TNFα biological agent, and if the level determined in step (a) is lower than the reference control level of expression, the patient is treated with an anti-TNFα biological agent.

4. The in vitro method of selecting a treatment for a rheumatoid arthritis patient according to claim 3, comprising:
a) determining the level of expression of PIK3CD, and additionally determining the level of expression of CX3CL1 in a body tissue test sample of the patient; and
b) comparing the level of expression of PIK3CD of step (a) with its reference control level of expression, and additionally comparing the level of expression of CX3CL1 of step (a) with its reference control level of expression, wherein if the level of expression determined in step (a) for PIK3CD is higher than its reference control level of expression and the level of expression determined in step (a) for CX3CL1 is lower than its reference control level of expression then the patient is not treated with an anti-TNFα biological agent, and if the level of expression determined in step (a) for PIK3CD is lower than its reference control level of expression and the level of expression determined in step (a) for CX3CL1 is higher than its reference control level of expression, then the patient is treated with an anti-TNFα biological agent.

5. An in vitro method of selecting a treatment for a rheumatoid arthritis patient, the method comprising:
a) determining the level of expression of PIK3CD, and additionally determining the level of expression of CX3CL1 in a body tissue test sample of the patient; and
b) calculating the ratio given by the level of expression of CX3CL1 over the level of expression of PIK3CD, wherein if said ratio is higher than a reference control ratio, then the patient is treated with an anti-TNFα biological agent, and if the ratio is lower than a reference control level then the patient is not treated with an anti-TNFα biological agent.

6. The method according to claim 1, wherein the body tissue test sample of the patient is selected from the group consisting of synovial tissue or fluid, plasma, blood serum or whole blood.

7. The method according to claim 1, wherein the level of expression is determined at the mRNA level.

8. The method according to claim 7, wherein the determination of the mRNA level of PIK3CD or the determination of the mRNA level of CX3CL1 comprise a step wherein a nucleic acid probe is hybridized to the mRNA of PIK3CD or to the mRNA of CX3CL1 respectively.

9. The method according to claim 8, wherein the determination of the mRNA level of PIK3CD comprises the use of a probe with the SEQ ID NO.1, and wherein the determination of the mRNA level of CX3CL1 comprises the use of a probe with the SEQ ID NO.2.

10. The method according to claim 1, wherein the level of expression is determined at the protein level.

11. The method according to claim 10, wherein the determination of the protein level of PIK3CD or the determination of the protein level of CX3CL1 comprise a step wherein an antibody or a fragment thereof binds to the protein coded by the PIK3CD gene or to the protein coded by the CX3CL1 gene respectively.

12. A kit for selecting a treatment for a rheumatoid arthritis patient, which comprises at least one agent for determining the level of expression of PIK3CD.

13. The kit according to claim 12, wherein the at least one agent for determining the level of expression of PIK3CD is capable of hybridizing to a partial sequence of the PIK3CD mRNA.

14. The kit according to claim 12, wherein the at least one agent for determining the level of expression of PIK3CD is an antibody or a fragment thereof which is capable of binding to the protein coded by the PIK3CD gene.

15. The kit according to claim 12, further comprising at least one agent for determining the level of expression of CX3CL1.

16. The kit according to claim 15, wherein the at least one agent for determining the level of expression of PIK3CD and the at least one agent for determining the level of expression of CX3CL1 are capable of hybridizing to a partial sequence of the PIK3CD mRNA and to a partial sequence of the CX3CL1 mRNA respectively.

17. The kit according to claim 15, wherein the at least one agent for determining the level of expression of PIK3CD and the at least one agent for determining the level of expression of CX3CL1 are antibodies or fragments thereof which are capable of binding to the protein coded by the PIK3CD gene and to the protein coded by the CX3CL1 gene respectively.

18. The kit according to claim 15, further comprising electronic means for calculating the level of expression of CX3CL1 and the level of expression of PIK3CD, and optionally for calculating the ratio given by the level of expression of CX3CL1 over the level of expression of PIK3CD.

19. The method according to claim 1, wherein the anti-TNFα biological agent is selected from the group consisting of infliximab, adalimumab, golimumab, certolizumab pegol, and etanercept.
